(19) United States
(12) Patent Application Publication
(10) Pub. No.: US 2024/0299622 A1
(43) Pub. Date: Sep. 12, 2024

(54) BIPHASIC OSTEOTENDINOUS REPAIR SCAFFOLDS

(71) Applicant: CASE WESTERN RESERVE UNIVERSITY, Cleveland, OH (US)

(72) Inventors: Phillip MCCLELLAN, Cleveland Heights, OH (US); Ozan AKKUS, Shaker Heights, OH (US); Michael MASTRAN, Tallmadge, OH (US); Subbakrishna SHANKAR, Cleveland, OH (US)

(21) Appl. No.: 18/597,085

(22) Filed: Mar. 6, 2024

Related U.S. Application Data
(60) Provisional application No. 63/488,632, filed on Mar. 6, 2023.

(51) Int. Cl. A61L 27/24; A61L 27/12; A61L 27/36; A61L 27/38; A61L 27/46

(52) U.S. Cl. CPC A61L 27/24; A61L 27/12; A61L 27/3687; A61L 27/3834; A61L 27/46; A61L 2300/64; A61L 2430/02; A61L 2430/10

(57) ABSTRACT

A biphasic osteotendinous repair scaffold is disclosed. The biphasic osteotendinous repair scaffold comprises a first phase comprising electrochemically aligned collagen (ELAC) threads and a calcium phosphate mineral and having a major surface. The biphasic osteotendinous repair scaffold also comprises a second phase comprising ELAC threads and having a major surface. The ELAC threads of the first phase are braided and crosslinked. The ELAC threads of the second phase are braided and crosslinked. The ELAC threads of the first and second phases form first and second interconnected macroporosities throughout the first and second phases, respectively. The calcium phosphate mineral of the first phase is distributed on the major surface of the first phase and within the interconnected macroporosity of the first phase. The second phase is adjacent to the first phase.

### FIELD OF THE INVENTION

The present invention relates generally to biphasic osteotendinous repair scaffolds, and more particularly to biphasic osteotendinous repair scaffolds comprising a first phase comprising electrochemically aligned collagen (ELAC) threads and a calcium phosphate mineral and having a major surface, and a second phase comprising ELAC threads and having a major surface.

### BACKGROUND OF THE INVENTION

Soft tissues such as ligaments or tendons often rupture at their interface with bone. Attachment of soft tissues to bone footprint is challenging for various reasons. First, the soft tissues retract and re-apposing them on the bone footprint may require too much tension. In this context, a bridging material to traverse that gap would be helpful. Second, soft tissues have evolved such that molecules in their biological compositions prevent mineral crystals from growing. Soft tissues thus integrate with bone tissue very slowly, through formation of a scar tissue which slowly and only partially remodels into a cohesive bone-soft tissue transition. This is why reattachment of soft tissue such as ligaments or tendons to bone result in re-ruptures on a high percentage of patients. These failures can occur in many locations in the body including, among others, the rotator cuff (RC) at the shoulder, Achilles tendon at the heel, and ligaments of the knee.

The social and economic burden of rotator cuff (RC) defects is highly significant. Tendons and muscles of the RC complex are critical to movement of arms. Full or partial thickness tears of RC occur because of acute trauma, repetitive occupational use, or degeneration during aging. Essential functions such as reaching out to shelves or tooth-brushing are compromised along with impairment in the quality of life (7, 8). There are 1.2 million physician visits for RC problems annually, and 330,000 RC procedures/year in the U.S. (98 per 100,000) at an estimated economic burden of $3.8 billion in 2019 (9).

There is an unmet need for effective treatment of patients with irreparable RC tears. Early stage small-sized tears can be treated by rest, physical therapy, or cortisol injections to provide symptomatic relief (10). Mid- to large tears are generally repaired by arthroscopic surgery to reattach torn segment to bone footprint on the humeral head by suture anchors (11). Some large tears may require overlay reinforcement patches (allogeneic, xenogeneic, or polymeric); however, their efficacy is not well-proven because evidence comes from lower-level case series generally with limited sample sizes (12).

Our interest lies in the repair of irreparable tears wherein muscle atrophy and fatty infiltration is pronounced, and the musculotendinous complex is retracted notably, making it impossible to perform primary repair by opposing the tendon on the bone-footprint without creating a damaging level of tension that would result in suture pull-out. This is an unmet need whose fulfillment requires a gap bridging biomaterial that will also connect the remnant musculotendinous complex to bone-footprint.

Subacromial spacers, such as the INSPACE balloon device, have found some acceptance in providing some relief, but a very recent double-blind, randomized and multi-center clinical trial concluded that the INSPACE balloon device was not more effective than debridement alone (13). Otherwise, irreparable tears remain outside the domain of arthroscopic surgery and often call for open repair.

There are no off-the-shelf biomaterial solutions that address irreparable tears.

Autografts do not have widespread use in repair due to lack of suitably sized donor locations. Allografts and xenografts may elicit immune response, scar tissue formation, or disease transmission (12, 14). Decellularized grafts also lack a connected open pore structure. Thus, cellular infiltration is highly restricted. Some patients are treated by invasive muscle transfer procedures (15). Otherwise, most patients live with limited mobility and chronic pain, eventually undergoing total joint arthroplasty (1, 2).

A regenerative platform for treatment of irreparable tears needs to fulfill the following: 1) Bulk tendon regeneration: Muscle retraction results in a gap between tendon and bone which needs to be bridged by off-the-shelf product that should drive regeneration of neo-tendon. 2) Surgical Handling: The scaffold should retain sutures to be attached to tendon proximally and to bone distally. 3) In vivo durability: The scaffold should bear functional loads in vivo. 4) Integration with bone: The distal end of the scaffold should be osteoconductive to accommodate bony ingrowth.

Monophasic scaffolds do not address the chronic RC degeneration problem adequately. Existing strategies generally address either bone or tendon aspects, whereas a chronic degeneration situation requires reconstitution of both domains. The delivery of osteoinductive factors to bone-tendon interface, for example, injectable calcium phosphates (16, 17), collagen gel/sponge/paste (18-20), etc., have reportedly resulted in increased strength and stiffness (18-21), and increased ossification (17, 22). Delivery of cytokines that promote connective tissue, such as GDF5 (23), CDMP2 (24), TGFβ 1-3 (25-28), and FGFs (28-31), have been reported to increase strength (23, 24, 26, 31), collagen output (23, 25), or collagen fiber organization (25, 27, 31). There are limitations to these studies, though, including the following: 1) short follow up period (<4 weeks), 2) beneficial outcomes not sustained at longer time points, 3) lack of characterization of tendon-specific markers, such as scleraxis or tenomodulin, for assessment of endpoint healing, and 4) generally neither a gap is imposed nor a chronic degeneration is induced. They do not model the irreparable condition sufficiently.

Moreover, to the best of our knowledge, only two research studies have used multiphasic scaffolds in vivo for RC repair; and only in an acute tear context as an overlay patch (47) or as a sheet (48) insertion between bone and tendon. These approaches are useful to treat acute tears where native tendon bears the load. However, chronic degeneration requires full load bearing scaffolds that address both tendon and bone regeneration.

Thus, a need exists for a bioinductive scaffold for regenerating bulk tendon volume to enable patients to sustain daily necessities of life.

### BRIEF SUMMARY OF THE INVENTION

A biphasic osteotendinous repair scaffold is disclosed. The biphasic osteotendinous repair scaffold comprises a first phase comprising electrochemically aligned collagen (ELAC) threads and a calcium phosphate mineral and having a major surface. The biphasic osteotendinous repair scaffold also comprises a second phase comprising ELAC threads and having a major surface. The ELAC threads of the first phase are braided and crosslinked. The ELAC threads of the second phase are braided and crosslinked. The ELAC threads of the first and second phases form first and second interconnected macroporosities throughout the first and second phases, respectively. The calcium phosphate mineral of the first phase is distributed on the major surface of the first phase and within the interconnected macroporosity of the first phase. The second phase is adjacent to the first phase.

In some embodiments, the ELAC threads of the first phase are twisted to form a yarn, the ELAC threads of the second phase are twisted to form a yarn, the yarn of the first phase is braided, thereby forming the ELAC threads of the first phase that are braided, and the yarn of the second phase is braided, thereby forming the ELAC threads of the second phase that are braided. In some of these embodiments, the yarn of the first phase comprises a 4-ply yarn and a 2-ply yarn, the 4-ply yarn of the first phase is used as triaxial threads within the first phase, and the 2-ply yarn of the first phase is used as oblique threads within the first phase, and the yarn of the second phase comprises a 4-ply yarn and a 2-ply yarn, the 4-ply yarn of the second phase is used as triaxial threads within the second phase, and the 2-ply yarn of the second phase is used as oblique threads within the second phase. Also in some of these embodiments, three or more concentric layers of the yarn of the first phase are braided on top of each other successively, and three or more concentric layers of the yarn of the second phase are braided on top of each other successively.

In some embodiments, the ELAC threads of the first phase are braided as monofilaments, and the ELAC threads of the second phase are braided as monofilaments.

In some embodiments, the scaffold has a load-bearing axis, and a majority of the ELAC threads are positioned along the load-bearing axis.

In some embodiments, the ELAC threads of the first and second phases are crosslinked with an iridoid crosslinking agent. In some of these embodiments, the iridoid crosslinking agent comprises one or more of genipin, loganin aglycone, oleuropein aglycone, or E-6-O-methoxycinnamoyl scandoside methyl ester aglycone. In some of these embodiments, the iridoid crosslinking agent comprises genipin.

In some embodiments, the calcium phosphate mineral comprises osteoinductive hydroxyapatite crystals or dicalcium phosphate dihydrate crystals.

In some embodiments, the second phase is adjacent to the first phase based on being contiguous with the first phase.

In some embodiments, the second phase is adjacent to the first phase based on being attached directly or indirectly to the first phase.

In some embodiments, the scaffold further comprises mesenchymal stem cells seeded on the ELAC threads of the first phase. In some of these embodiments, the mesenchymal stem cells are autologous mesenchymal stem cells. Also in some of these embodiments, to the extent that human or animal cells other than mesenchymal stem cells are present on the ELAC threads of the first phase, more mesenchymal stem cells are present than the other human or animal cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a perspective view of a biphasic osteotendinous repair scaffold as disclosed herein.

FIG. 2 shows a side view of the biphasic osteotendinous repair scaffold of FIG. 1.

FIG. 3 shows a sectional view of a first phase of the biphasic osteotendinous repair scaffold of FIG. 2.

FIG. 4 shows a sectional view of a second phase of the biphasic osteotendinous repair scaffold of FIG. 2.

FIG. 5 includes parts (A)-(F). Part (A) shows a schematic depiction of electrochemical alignment and compaction of collagen molecules (green rods) at the isoelectric point between linear electrodes to form electrochemically aligned collagen (ELAC) threads. Part (B) shows an ELAC thread, in blue, formed between an anode and a cathode. Part (C) shows results of compensated polarization confirming that collagen molecules in ELAC threads and tendon are oriented along their longer axes, manifested in blue. Part (D) shows a rotating electrode wheel (left side of image) transforming collagen solution applied on top by a syringe (not shown) to ELAC thread. Part (E) shows ELAC thread on a spool. Part (F) shows a scaffold fabricated by braiding as described and shown in FIG. 11 and FIGS. 13-15.

FIG. 6 includes parts (A-D). Parts (A) and (B) indicate that focal adhesions of talin-GFP transfected cells, shown in green, on random collagen (A) and ELAC (B, yellow double headed arrow: collagen thread alignment axis) show more prominent establishment of focal adhesions on ELAC than on random collagen. Parts (C) and (D) indicate that actin cytoskeletons on ELAC (D) was aligned parallel to longer axis of threads, whereas actin cytoskeletons of cells seeded on random collagen were disorganized (C).

FIG. 7 shows results for gene expression of cells seeded on ELAC normalized by that on random collagen (p<0.05 for every gene at both time points).

FIG. 8 shows that mesenchymal stem cells (also termed MSCs) seeded on ELAC show strong presence of tenomodulin (day 14), as revealed by immunostaining, in blue, without addition of tenoinductive factors in culture media.

FIG. 9 includes parts (A)-(F). Part (A) shows that host collagen (stained in blue) is present in and around the collagen scaffold (white border). Parts (B-D) are higher magnification images showing individual threads (*, red) and host collagen deposition around the threads (blue). Part (E) shows that polarized picrosirius red imaging demonstrates that host collagen is oriented parallel to collagen threads (*) as depicted by birefringence. Part (F) shows that immunohistochemical staining (*, purple) confirmed the presence of collagen-I and tendon marker tenomodulin in host-made tissue in and around the scaffold (negative control stains were absent for these markers, data not shown).

FIG. 10 shows repair strength of injured rabbit infraspinatus normalized by the intact contralateral. Segmental defects repaired by ELAC attained similar strength with the positive control Direct Repair.

FIG. 11 includes parts (A) and (B), showing industrial braiding of spooled collagen yarns (A) as a scaffold (B).

FIG. 12 includes parts (A)-(E). Part (A) shows that hydroxyapatite (also termed HA) integrated ELAC threads are positive for alizarin stain. Part (B) shows that mesenchymal stem cells seeded on HA-ELAC threads and cultured in osteoinductive media in vitro were positive for Runx2 indicating that HA-ELAC threads are osteoconductive. Part (C) shows a 3D-microCT scan of the osteoconductive segment of baseline scaffold a priori to implantation. Inset shows typical appearance of mineralized threads in the plane through dashed line. Part (D) shows the same scaffold at 3 months after being onlaid on rabbit humerus. Part (E) shows that the area fraction of mineral increased ˜3-fold (p<0.05, each bar is average of 5 transverse sections taken along the scaffold at the baseline, and at 3 months).

FIG. 13 shows an OsTend scaffold that consists of a calcium-phosphate mineral crystal impregnated distal end (stereomicrograph shown in red inset) and a mineral free tenoconductive segment (blue inset).

FIG. 14 shows a microCT scan of section a-a of the distal end of the OsTend scaffold of FIG. 13 showing penetration of mineralization to the core of the scaffold and through the full thickness of threads.

FIG. 15 shows results of a Raman spectrum identifying mineral deposits resulting from an alternative soaking process to be of hydroxyapatite origin based on the presence of phosphate peaks.

FIG. 16 shows microCT reconstruction of rabbit infraspinatus muscle (red) demonstrating fatty infiltration (yellow) secondary to detachment of tendon at the enthesis.

### DETAILED DESCRIPTION OF THE INVENTION

The biphasic osteotendinous repair scaffold disclosed herein, having a structure comprising two adjacent phases, should provide an effective treatment for patients with irreparable RC tears.

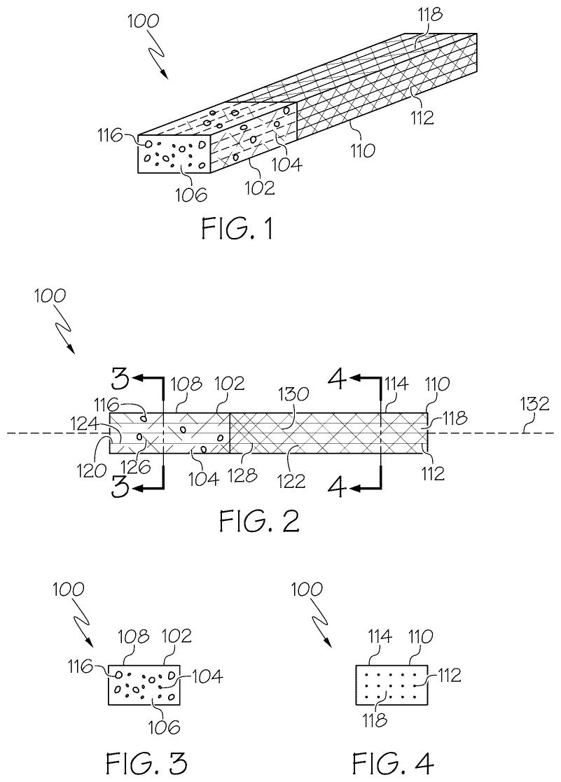

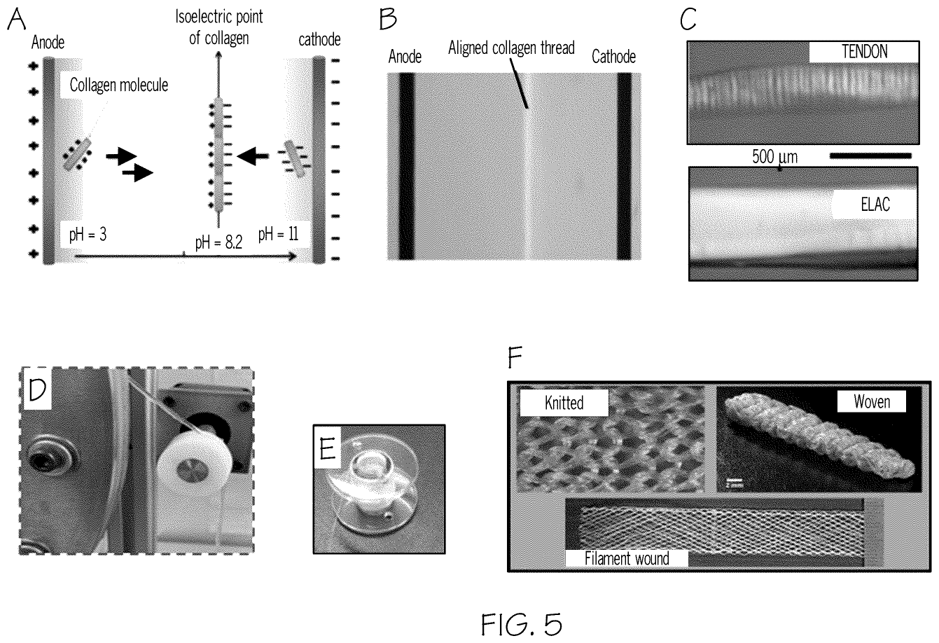

An exemplary biphasic osteotendinous repair scaffold 100 is shown in FIGS. 1-4.

The biphasic osteotendinous repair scaffold 100 comprises a first phase 102 comprising electrochemically aligned collagen (ELAC) threads 104 and a calcium phosphate mineral 106 and having a major surface 108, and a second phase 110 comprising ELAC threads 112 and having a major surface 114. The ELAC threads 104 of the first phase 102 are braided and crosslinked. The ELAC threads 112 of the second phase 110 also are braided and crosslinked. The ELAC threads 104, 112 of the first and second phases 102, 110 form first and second interconnected macroporosities 116, 118 throughout the first and second phases 102, 110, respectively. The calcium phosphate mineral 106 of the first phase 102 is distributed on the major surface 108 of the first phase 102 and within the interconnected macroporosity 116 of the first phase 102. The second phase 110 is adjacent to the first phase 102.

The first phase 102 is macroporous and mineralized prior to implantation into a patient (also termed pre-mineralized) to accommodate and promote ingrowth of bone at the major surface 108 of the first phase 102. The second phase 110 is macroporous to accommodate ingrowth of tendon at the major surface 114 of the second phase 110. As noted, the second phase 110 is adjacent to the first phase 102, for example based on being contiguous with the first phase 102, or being attached directly or indirectly to the first phase 102. During use, the first phase 102 is put in contact with bone of the patient at the major surface 108 of the first phase 102 by implantation. The second phase 110 is positioned to allow ingrowth of tendon. The duality of the structure of the biphasic osteotendinous repair scaffold 100 allows for hard tissue growth at the major surface 108 of the pre-mineralized first phase 102 of the biphasic osteotendinous repair scaffold 100 and soft tissue growth at the major surface 114 of the unmineralized second phase 110 of the biphasic osteotendinous repair scaffold 100, integrating the disparate hard and soft tissue types effectively.

In some embodiments, the calcium phosphate mineral 106 comprises osteoinductive hydroxyapatite crystals or dicalcium phosphate dihydrate crystals. As noted above, the first phase 102 of the biphasic osteotendinous repair scaffold 100 comprises the calcium phosphate mineral 106. The calcium phosphate mineral 106 can be formed as osteoinductive crystals, for example, osteoinductive hydroxyapatite crystals or dicalcium phosphate dihydrate crystals. This can be accomplished by repeated sequential soaking of a portion of a scaffold in a solution of calcium and a solution of phosphate. The molar ratios of the calcium and the phosphate in the solutions can be varied to determine the type of crystals formed.

The ELAC threads 104 of the first phase 102 and the ELAC threads 112 of the second phase 110 can be used directly as monofilaments and/or as a multi-ply yarns formed from multiple monofilaments, for example, 2-ply, 3-ply, 4-ply, or 5-ply yarns, formed from 2, 3, 4, or 5 monofilaments, respectively.

In some embodiments, the second phase 110 is contiguous with the first phase 102. In accordance with these embodiments, a biphasic osteotendinous repair scaffold 100 can be made by the following process. First, an initial uncrosslinked scaffold is formed from ELAC threads. For example, yarn can be formed from the ELAC threads. The yarn can be braided over a carrier mandrel, with two or more concentric layers, e.g., three concentric layers, being braided on top of each other successively. The braids can be cut into segments and flattened as a ribbon, e.g., each segment having a thickness, for example, of 1 to 5 mm, a width, for example, of 5 to 50 mm, and a length, for example, of 20 to 60 mm. These dimensions would be suitable for rotator cuff repair in human patients. These dimensions and others may be suitable for other applications too. Both ends of a segment can be fused with an acidic collagen solution to obtain the initial uncrosslinked scaffold. The initial uncrosslinked scaffold can then be treated with a crosslinking agent, such genipin, to form a crosslinked scaffold. Then a first portion of the crosslinked scaffold can be soaked in two mineralizing solutions, one being a calcium solution and the other being a phosphate solution. Repeated sequential soaking of the first portion in these two solutions with intermediate drying steps results in ongrowth and ingrowth of calcium phosphate mineral, for example hydroxyapatite crystals or dicalcium phosphate dihydrate crystals, in the first portion of the crosslinked scaffold that is dipped in the solutions, thereby forming the first phase 102. A second remaining portion of the crosslinked scaffold that is not soaked in the solutions remains unmineralized, and corresponds to the second phase 110. By this process, a single continuum of a biphasic osteotendinous repair scaffold 100 is obtained, a first phase 102 that is mineralized and a second phase 110 that is unmineralized, wherein the second phase 110 is contiguous with the first phase 102.

In some embodiments, the second phase 110 is attached to the first phase 102 based on being stitched or adhered to the first phase 102.

In some embodiments, the first phase 102 can be induced along end-to-end segments, and thus serially connected. The mineralized end of the series can then be inserted into a pocket that is machined in bone footprint and secured by a pin, sutures, or other methods for native bone to integrate.

Considering yarn formed from ELAC threads in more details, in some embodiments the ELAC threads 104 of the first phase 102 are twisted to form a yarn 120, the ELAC threads 112 of the second phase 110 are twisted to form a yarn 122, the yarn 120 of the first phase 102 is braided, thereby forming the ELAC threads 104 of the first phase 102 that are braided, and the yarn 122 of the second phase 110 is braided, thereby forming the ELAC threads 112 of the second phase 110 that are braided.

In some of these embodiments, the yarn 120 of the first phase 102 comprises a 4-ply yarn and a 2-ply yarn, the 4-ply yarn of the first phase 102 is used as triaxial threads 124 within the first phase 102, and the 2-ply yarn of the first phase 102 is used as oblique threads 126 within the first phase 102. Also in these embodiments, the yarn 122 of the second phase 110 comprises a 4-ply yarn and a 2-ply yarn, the 4-ply yarn of the second phase 110 is used as triaxial threads 128 within the second phase 110, and the 2-ply yarn of the second phase 110 is used as oblique threads 130 within the second phase 110.

Also in some of these embodiments, three or more concentric layers of the yarn 120 of the first phase 102 are braided on top of each other successively, and three or more concentric layers of the yarn 122 of the second phase 110 are braided on top of each other successively.

Alternatively or additionally, in some of these embodiments a single layer long braid can be folded to obtain a multilayer scaffold.

In some embodiments, the ELAC threads 104 of the first phase 102 are braided as monofilaments, and the ELAC threads 112 of the second phase 110 are braided as monofilaments.

As noted above, the biphasic osteotendinous repair scaffold 100 comprises a first phase 102 having a major surface 108 and a second phase 110 has a major surface 114. The major surfaces 108, 114 of the first and second phases 102, 110 can be, for example, top, bottom, and/or side surfaces of the biphasic osteotendinous repair scaffold 100.

In some embodiments, the first phase 102 forms a first layer having a major surface, the second phase 110 forms a second layer having a major surface, and the first layer is attached to the second layer across their respective major surfaces, thereby forming a bilayer. In accordance with this embodiment, the biphasic osteotendinous repair scaffold 100 can be implanted, for example, between bone and tendon, with the first layer facing the bone and the second layer facing the tendon. The biphasic osteotendinous repair scaffold 100 can then act as an intermediate layer between the bone and tendon and thereby serve its function.

In some embodiments, the biphasic osteotendinous repair scaffold 100 has a load-bearing axis 132, and a majority of the ELAC threads 104, 112 are positioned along the load-bearing axis 132.

In some embodiments, the ELAC threads 104, 112 of the first and second phases 102, 110 are crosslinked with an iridoid crosslinking agent. In some of these embodiments, the iridoid crosslinking agent comprises one or more of genipin, loganin aglycone, oleuropein aglycone, or E-6-O-methoxycinnamoyl scandoside methyl ester aglycone. In some of these embodiments, the iridoid crosslinking agent comprises genipin.

As noted above, the ELAC threads 104 of the first phase 102 are braided and crosslinked, and the ELAC threads 112 of the second phase 110 also are braided and crosslinked. In some embodiments, the ELAC threads 104, 112 are first braided, then crosslinked. In some embodiments, the ELAC threads 104, 112 are first crosslinked, then braided.

In some cases it may be advantageous to seed the biphasic osteotendinous repair scaffold 100 with mesenchymal stem cells to promote ongrowth and ingrowth of bone at the major surface 108 of the first phase 102. Thus, in some embodiments the biphasic osteotendinous repair scaffold 100 further comprises mesenchymal stem cells seeded on the ELAC threads 104 of the first phase 102. In some of these embodiments, the mesenchymal stem cells are autologous mesenchymal stem cells, i.e., mesenchymal stem cells derived from the patient into which the scaffold is to be implanted. Also in some of these embodiments, to the extent that human or animal cells other than mesenchymal stem cells are present on the ELAC threads 104 of the first phase 102, more mesenchymal stem cells are present than the other human or animal cells. For example, in some embodiments, to the extent that human or animal cells other than mesenchymal stem cells are present on the ELAC threads 104 of the first phase 102, at least 51%, at least 60%, at least 70%, at least 80%, at least 90%, at least 95%, at least 99%, or 100% of the total number of human or animal cells present on the ELAC threads 104 of the first phase 102 are macrophages.

### EXAMPLES

### Example 1: OsTend Scaffold

We developed our biphasic osteotendinous repair scaffold upon recognizing that monophasic ELAC scaffolds drive tendon-like regeneration in vivo, but provide limited regeneration of the enthesis (5, 6, 46).

For context, the process of electrochemical alignment and compaction (41) generates resilient ELAC threads that can be used to fabricate load-bearing collagen biotextiles (3, 5, 32-38). Type I collagen is the most abundant protein in tendon. Reconstituted collagen gels and sponges are mechanically unfit for replacing load-bearing tendon (39, 40). The ELAC process compacts and aligns collagen molecules as threads whose strength and modulus converge to that of native tendon (36). In the ELAC process, aqueous solution of collagen (medical grade bovine type I, Collagen Solutions Inc., 3 mg/mL) is applied between parallel line electrodes (20-40 V, FIG. 1(A)) between which a pH gradient forms (38). Ampholytic collagen molecules near the cathode become negatively charged while those near the anode assume positive charge, resulting in their repulsion. Molecules are compacted at the isoelectric point (pI) (FIG. 1(A)).

Convectional forces induce molecule alignment parallel to the longer axis of threads to a degree comparable to that of tendon (FIG. 1(A-B)). A rotating electrode electrochemical thread production device (FIG. 1(D)) generates threads in continuous length on a spool (FIG. 1(E)), which can then be used to fabricate biotextile scaffolds (3) (FIG. 1(F)). This biotextile format presents an interconnected macroporosity that facilitates rapid tissue ingrowth in vivo (42).

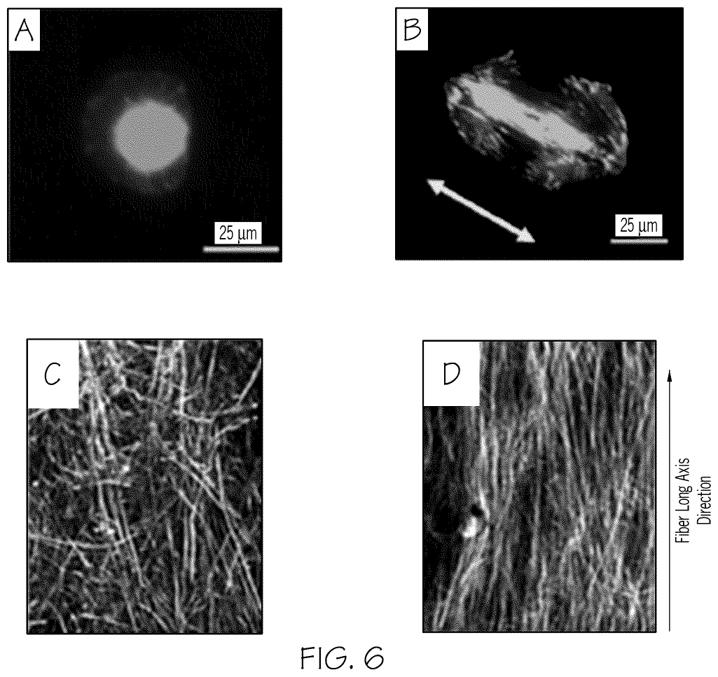

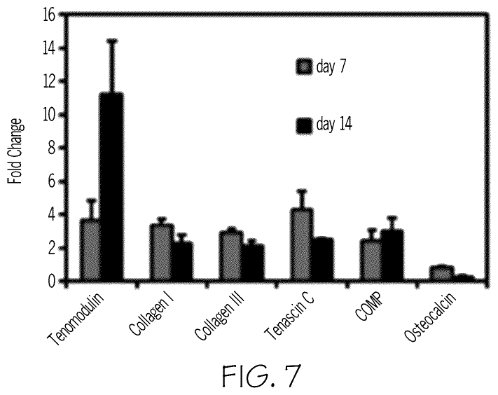

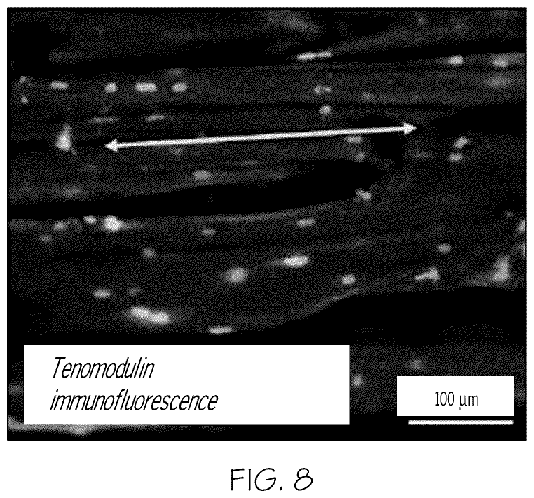

ELAC-induced cytoskeletal elongation promotes tenogenesis in vitro (3, 37, 43). Tenogenic potential of mesenchymal stem cells increases on anisotropic materials such as decellularized tendon (44), nanofibrous aligned PLLA (45), silk, and also using ELAC threads (FIG. 6(A-D), FIG. 7, and FIG. 8) (3, 4, 32, 37). Cytoskeletons of mesenchymal stem cells on ELAC threads elongate under topographical guidance, compared to those seeded on randomly-oriented collagen (FIG. 6(A) vs. FIG. 6(B)). The axis of elongation is parallel to the longer axis of the thread (FIG. 6(C) vs. FIG. 6(D)). Cells also form more prominent focal adhesions on ELAC (FIG. 6(A) vs. FIG. 6(B), data quantifiably published in (32)). On ELAC, mesenchymal stem cells express the gene for tendon-specific tenomodulin, 4- and 11-fold greater on days 7 and 14, respectively, than mesenchymal stem cells seeded on randomly oriented collagen (p<0.05) (37). Tendon-related markers also display increased gene expressions (37) on ELAC (all at p<0.05, FIG. 7). Besides gene expression, production of tenomodulin is evident on ELAC by immunofluorescence (FIG. 8), driven topographically in a growth medium that is absent for tenoinductive factors.

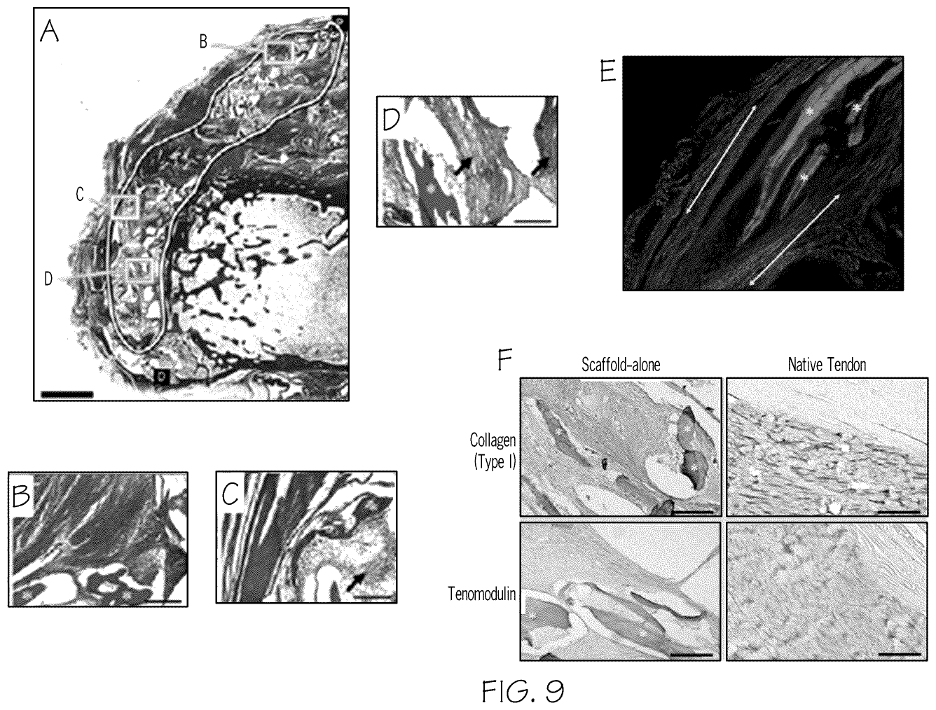

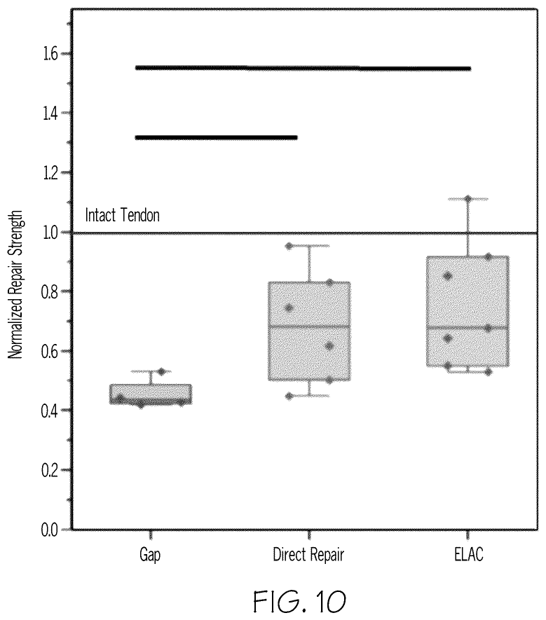

As noted above, monophasic ELAC scaffolds drive tendon-like regeneration in vivo, but provide limited regeneration of the enthesis (5, 6, 46). For example, we applied monophasic woven ELAC scaffolds to repair rabbit infraspinatus (New Zealand White) where 50% of the original tendon was resected distally and repaired. Woven scaffolds were sutured at the proximal end on the remnant tendon, and the distal end to the bone via bone tunnels to bridge the gap defect. Direct repair was included as the positive control where tendon is sharply dissected at enthesis and reattached without segmental defect induction. A gap defect no-repair group was included as the negative control. We assessed regeneration at 6 months (N=11/group, 8 biomechanics and 3 immuno/histology). The result was that Masson's trichrome sections indicated a dense de novo collagen deposition in and around the scaffold in close association with collagen threads (FIG. 9(A-D)). Alignment of de novo collagen that is produced by the host followed the template of collagen threads as revealed by picrosirius red staining (FIG. 9(E)). ELAC repair was strongly positive for tendon-specific marker tenomodulin and type-I collagen (FIG. 9(F)), whereas both markers were absent in the direct repair group for all animals (data shown in (6)). Normalized-strength (strength of operated shoulder divided by strength of intact contralateral) was significantly greater than the negative control gap group, and, was statistically similar to the positive control direct repair group (p<0.05) (FIG. 10). Median normalized strength for ELAC group was ˜70% of the intact shoulder. While the convergence between the failure loads of intact tendon and monophasic ELAC groups is a significant success, 30% of the strength remains to be recovered.

In mechanical tests involving a monophasic scaffold as presented in FIG. 10, the failure consistently occurred at the junction of the monophasic scaffold with bone.

Our biphasic osteotendinous repair scaffold, also termed OsTend scaffold, is intended to address the reconstitution of the enthesis between the scaffold and bone. We will attain a more robust integration at the enthesis by: a) improving the scaffold concept from a monophasic tendon construct to a biphasic osteotendinous repair format by introducing an osteoconductive distal end, b) by adopting braiding instead of weaving as the textile process to position threads predominantly along the load-bearing axis, and c) by modifying the surgical model to nest the osteoconductive end deeper into bone for enhanced anchorage.

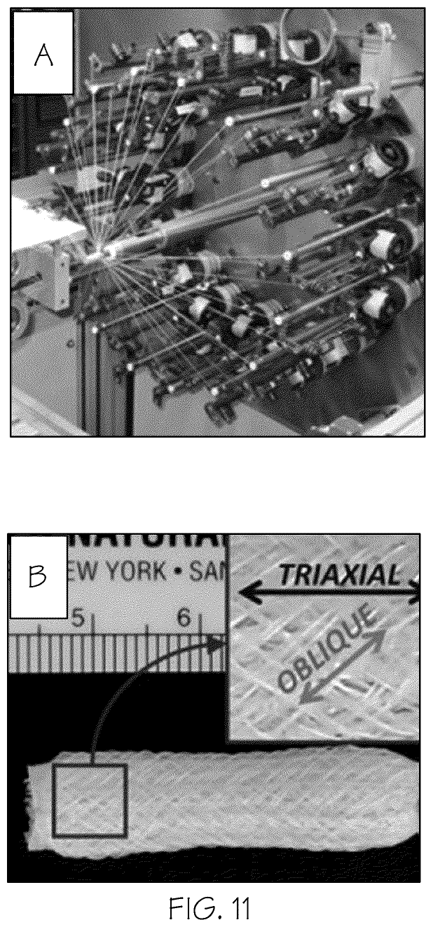

Biphasic OsTend scaffold: Spools of collagen threads can be loaded to industrial braiding machinery (Steegers Inc.) to obtain tape-shaped scaffolds with controlled macroporosity for cellular infiltration as shown in (FIG. 11). The scaffold is then crosslinked and the distal end region is infused with osteoinductive hydroxyapatite (HA) crystals as detailed in the Examples, section 2.1, below.

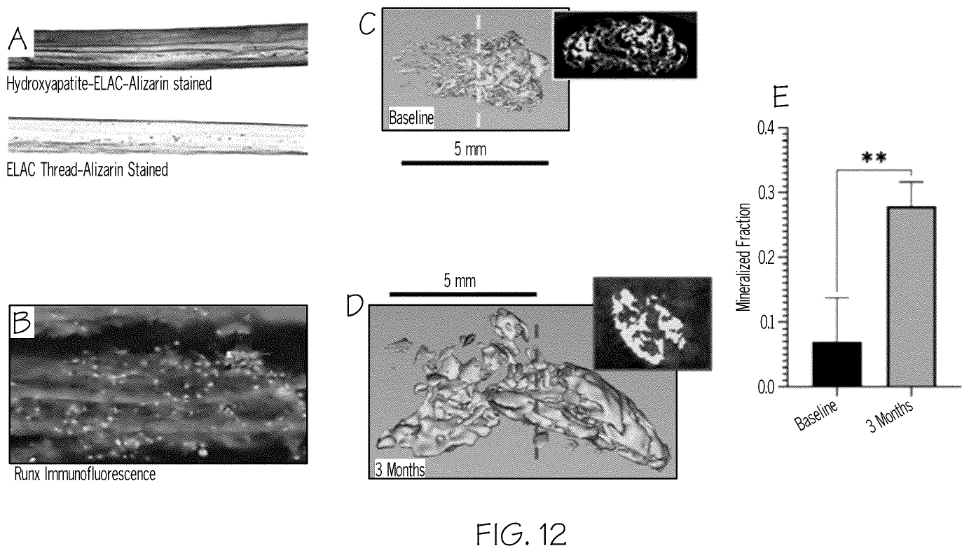

Osteoconductivity of HA-ELAC: Feasibility has been demonstrated as follows. Osteocalcin expression by mesenchymal stem cells seeded on ELAC is downregulated (FIG. 7). To bestow ELAC threads with osteoconductivity, HA crystals were grown on ELAC threads using an alternating soaking method as described in section 2.1 below to obtain HA-ELAC threads. HA presence is confirmed with alizarin staining (FIG. 12(A)). Mesenchymal stem cells were seeded on HA-ELAC threads cultured in osteogenic medium product Runx2. Thus, HA-ELAC threads are osteoconductive in vitro (FIG. 12(B)).

OsTend scaffold was implanted on a segmental defect in rabbit infraspinatus in vivo as described above to assess its osteoconductivity. The osteoconductive distal end was on-laid on the tendon footprint with subchondral bone shaved off to provide access to the marrow stock. We have not inserted the distal end in the humeral head to be able to distinguish mineralizing OsTend scaffold from the native trabecular bone. The same implant was scanned by MicroCT at the baseline, and later at three months after sacrifice. Five equidistant image-sections were obtained from each scan transversely to the longer axis of the osteoconductive segment of the scaffold (FIG. 12(C-E)). The area fraction of mineralized regions increased substantially in vivo, suggesting that there is de novo osteogenesis around mineralized threads. No ectopic calcification was observed in the tendinous segment. This pilot animal in which we followed the same scaffold longitudinally shows the feasibility of ossification in OsTend scaffold site-specifically.

Presently, biomaterials that are used in RC repair clinically are not suitable for irreparable defects. Dermal patches serve a passive mechanical reinforcement function that may be beneficial in augmentation of small to mid-sized tears. However, irreparable tears necessitate regenerative cues that are largely absent in decellularized allografts or xenografts. To the best of our knowledge, there are no biphasic scaffolds in clinical practice that will result in tendon regeneration while integrating with bone effectively.

Our fully-load bearing, defect bridging, suture retaining, structurally multiphasic, compositionally multiphasic, and bioinductively multiphasic regeneration OsTend scaffold disclosed herein offers a way to address the unmet need.

Our OsTend scaffold also may impact other soft-hard tissue regeneration challenges such as ligament-bone, intervertebral disc-endplate, and osteochondral repair.

Our OsTend scaffold's translational potential is significant as well. The manufacturing process can be scaled-up as shown by proof-of-concept braiding run that was accomplished at Steeger Inc. (FIG. 11). A preclinical large animal model (e.g. ovine) may be pursued.

### Example 2: Testing OsTend Scaffold for Improvement of Repair Outcomes

### Section 2.0: Approach

Aim 1: Demonstrate that the OsTend scaffold strategy improves repair outcomes in irreparable defect of rotator cuff.

### Section 2.1: Scaffold Fabrication

Thread fabrication: Threads will be fabricated as explained above. Four threads will be twisted to obtain a 4-ply yarn to be used as triaxial threads. Two threads will be twisted as 2-ply yarn to be used for oblique threads (FIG. 11(B), inset). Braiding: Braiding will be performed at a contract manufacturer (Steeger, Inman, SC). Yarns are braided over a carrier mandrel, and three concentric layers will be braided on top of each other successively. Each layer will have 16 triaxial 4-ply yarns, resulting in a total of 48 load-bearing triaxial yarns. Each 4-ply triaxial yarn is 3 N in terms of wet-strength and for a total of 48 triaxial yarns, the strength is estimated at 144 N which matches the strength of native rabbit infraspinatus tendon under identical testing conditions (129.6±24.9 N (46)). Braids will be cut into 25 mm long segments, flattened as a ribbon, and both ends will be fused with acidic collagen solution to obtain the implant in uncrosslinked form as shown in FIG. 11(B). Total article thickness is 3 mm, has a width of 6 mm and length of 25 mm.

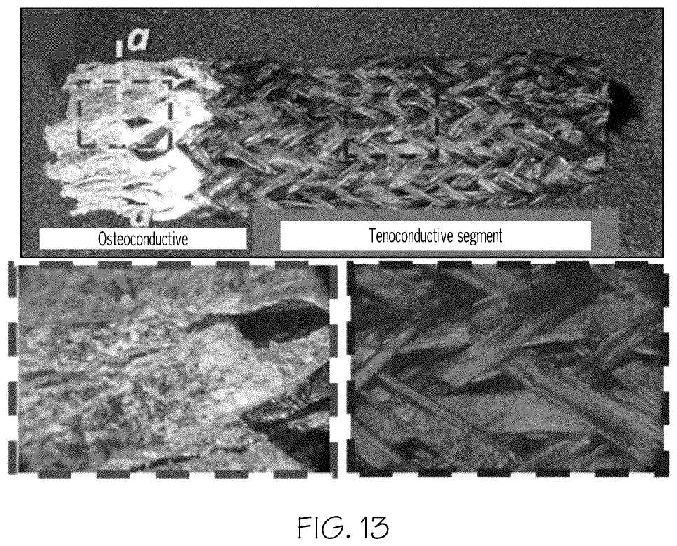

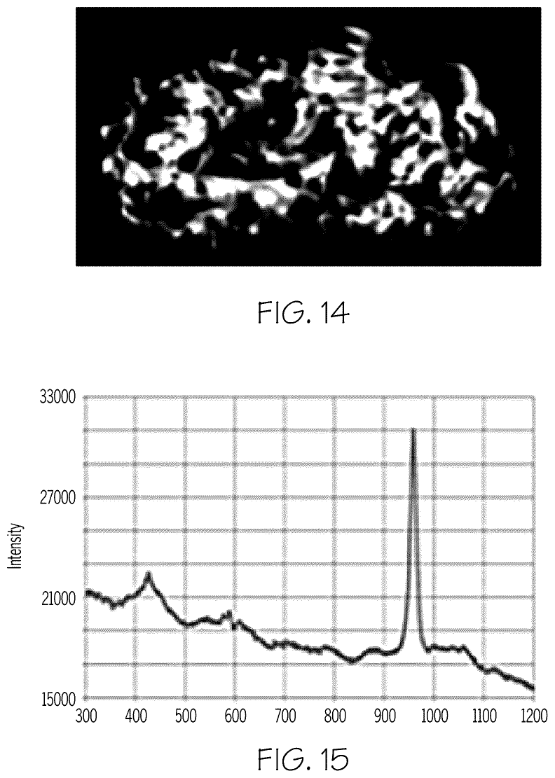

Crosslinking and HA-Infusion: Braiding is performed by using uncrosslinked dry collagen threads. To provide strength in wet state, braided article is crosslinked in 0.625% genipin-ethanol solution for 3 days at room temperature as we described earlier (36). The next step involves growth of HA crystals in the 5 mm long distal segment by alternating soaking (AS) (FIG. 13). During AS, scaffold's end region is first dipped in calcium (240 mM CaCl₂) solution buffered to a pH of 7.4 by Tris base), removed, and then dipped in phosphate (120 mM NaH2PO4) solutions. This cycle is repeated for 5 times. Surface macrographs (FIG. 13) and in-depth microCT scans (FIG. 14) confirm the presence of mineral deposits across the continuum of the distal segment of the scaffold through AS. Raman spectrum of grown deposits confirm the presence of hydroxyapatite crystal stoichiometry based on the location of the symmetric stretch phosphate peak at 958 cm-1 (FIG. 15). Implants will be sterilized by EtO (37° C., 50% Humidity, 50 mbar, 240 minutes, Steris).

### Section 2.2: Rabbit Infrasupinatus Tendon Chronic Degeneration (RIC) Model

Overview and Justification: Rabbit infraspinatus is nominally macroscale (mms in thickness and width, a few centimeters in length). Authoritative reviews on animal models of tendon repair list rabbit in the large animal model group (49). RIC model is also well-established in the prior tendon tissue engineering literature (50, 51). Rabbit RC is similar to human RC in certain regards as elucidated by others (52). Finally, rabbit model offers to test our hypothesis with sufficient statistical power affordably.

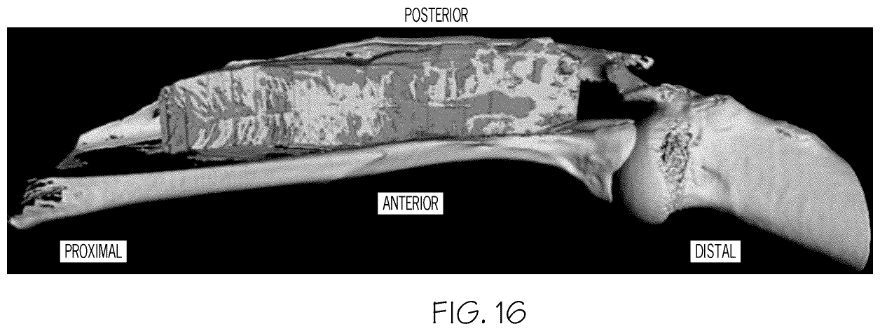

Surgical model: RIC model involves a two-step surgery. In the first stage surgery, the tendon is fully detached at the enthesis and the animal recovers for 6 weeks. During this period of detachment the muscle tendon complex retracts, and, is also subjected to fatty infiltration as we reported earlier (53) (FIG. 16). These are basic hallmarks of chronic degeneration observed in irreparable defects. Following six weeks, the second stage surgery will repair the defect site by grafting the scaffold between the retracted musculotendinous complex and bone where inverted mattress sutures are secured to the humerus via two 1 mm diameter holes that are 5 mm apart. In attaching the implant to bone, a 5 mm deep trough will be introduced in the bone to nest the entire 5 mm mineralized distal end of the scaffold within bone's continuum as illustrated in the Aim page. Surgeries will be performed unilaterally with the contralateral as the positive control.

### Section 2.2.1: Treatment Groups and Time-Points

We will compare reconstitution of enthesis under three groups: biphasic OsTend scaffold repair (OT), repair performed by monophasic scaffold (MO, pure collagen scaffold without HA deposition on distal end), and intact contralaterals (IC, positive control) will be the third group implicitly. There will be two time points of 2 weeks and 12 weeks. Previously we observed both 3 month (5) and 6 month (54) time points and healing strength was comparable; thus, 3-months is chosen as the end-point. We will operate on 12 rabbits/group/time point (N=48, equally distributed as male and female New Zealand White Rabbits). Scaffolds from 8 rabbits/group will be used for mechanical testing and the remaining four will be used to quantify healing immunohistologically.

In vivo assessment of joint healing biomechanics: Joint strength and joint range of motion will be measured as functional repair outcomes in vivo. Measurements will be performed longitudinally over time for each rabbit: 1) healthy reference baseline prior to the first surgery before induction of detachment, 2) immediately after first surgery after detachment to record detached baseline, 3) at 6 weeks at the end of the chronic degeneration period before the repair is performed as chronic baseline, 4) at 6 weeks after the repair surgery is performed, 5) after the repair surgery prior to the sacrifice. Infraspinatus muscle contractions will be induced using stimulation while the rabbit is already under anesthesia to assess shoulder strength and range of motion. Charge-balanced, cathodic stimulation will be delivered through stimulating electrodes (TECA Monopolar Needle Electrodes #902-DMG75-TP) inserted at the suprascapular notch by a custom-made programmable stimulator. To assess strength, the forelimb will be fixed in a stereotactic frame with the paw secured to a load cell while stimulation is delivered, as done in feline studies (55). Trains of 15 short tetanic bursts (1 second each separated by 5 seconds at 100 Hz, 5 mA 56) will be used to obtain mean force±standard deviations. Stimulus intensities that evoke 50% of the maximum force (as measured prior to induced degeneration during Surgery #1) will be used for comparison across time points to avoid reinjury of the healing tendon. ROM will be assessed using motion capture (Dual camera 3D Image system. Correlated Solutions Inc.) while the forelimb moves freely in response to both passive manual stretching and active contraction from the same stimulation protocol. Because each animal will be followed longitudinally, a paired-test will be used (Wilcoxon or paired t-test if normal) to assess convergence to healthy reference baseline, or improvement from detached/chronic baselines.

An experimental design for animal studies is provides in TABLE 1. This experimental design involves testing two time points, 2 weeks and 12 weeks, along with quantifying intact controls as the baseline.

TABLE 1
Experimental design for animal studies.

| Intact* | OsTend Scaffold | | | | Monophasic Scaffold | | | |
|---|---|---|---|---|---|---|---|---|
| Baseline | 2 Weeks | | 12 Weeks | | 2 Weeks | | 12 Weeks | |
| M | H | M | H | M | H | M | H | M |
| 8 | 4 | 8 | 4 | 8 | 4 | 8 | 4 | 8 |

H: Immuno/histological; M: Mechanical; * H for intact group is available from prior studies as embedded blocks; N=12/group/time point for joint mechanics and microCT.

### Section 2.2.2: Quantification of Repair Status

Fatty infiltration: Scapulohumeral complexes will be dissected bilaterally and subjected to micro-CT (Siemens Inveon, 75-80 kV, 470-500 pa, 630-670 ms exposure, 21 μm voxel edge lengths). Gray level intensity will be calibrated by using phantom tissues. CT data will be processed (Matlab) to measure fatty infiltration in the muscle as we have done previously (53) (FIG. 16).

Bone volume: Cortical thickness, trabecular thickness and BV/TV at the humeral head will be quantified using CT scans at two locations: a) repair site (i.e. the former trough that is filled with the distal segment of OsTend scaffold), b) lateral to the repair site to determine effects of repair remotely at the humerus.

Mechanical Testing: The length, width, and thickness of the regenerated tendon portion will be measured by custom-made electrical contact sensors. Bone-tendon attachment region will be 3D-scanned using a stereoimaging system (Correlated Solutions) from which footprint attachment area will be measured. Samples will be gripped at the muscle using a clamp cooled with liquid nitrogen. Humeral shaft will be potted in PMMA. Samples will then be preconditioned at 5 N amplitude cyclic load for 50 cycles, and loaded to failure (5 mm/sec). Load-displacement curves will be recorded to obtain the failure load and deformation. Stiffness will be calculated using linear regression. Failure strength and location will be recorded. Ultimate stress & strain and modulus will be calculated by using the apparent area depending on failure location (generally enthesis, sometimes mid-substance). Baseline repair strength will be measured by (N=8/group) implanting scaffolds in cadaveric rabbit shoulders using described surgical procedures and testing mechanically.

### Section 2.2.2.1: Tendon Regeneration

Quantitative Histology: Samples will be fixed in neutral buffered formalin and demineralized in EDTA for quantitative histomorphometry. Sections will be prepared for histology. Volume fractions of ELAC threads and de novo collagen deposition will be measured by point counting of Masson's trichrome stained sections where acellular ELAC threads can be easily distinguished from de novo fibrous tissue (5, 6). Picrosirius stained sections will be imaged under cross polarization (Olympus BX51) to obtain the fraction of fibers oriented parallel to the longer axis of the tendon as before (57). Immunohistology will be performed for Type I/III collagen, tenascin-C, tenomodulin, and decorin. Based on a systematic review of tendon repair scoring methods (58), Watkins method (59) is accepted as one of the more complete scoring systems. This method involves composite evaluation of scores for cellularity, cell morphology and collagen alignment to obtain a summative score wherein higher sums represent proximity to a tendon-like tissue.

### Section 2.2.2.2: Enthesis Regeneration

Safranin-O stain will be used to label fibrocartilage. Histological scoring will be as defined by Yokoya et al. (51) which has been used by several other groups (28, 48). Scoring criteria are: continuity at insertion (C), alignment of fibrous tissue (R), presence of fibrocartilage (F), presence of tidemark (T). C+R−F−T− is scored as 1, C+R+F−T− is scored as 2, C+R+F+T− is scored as 3, and C+R+F+T+ is scored as 4. Using ImageJ software, total area of fibrocartilage phase will be traced. Picrosirius red sections will be imaged under circular polarization to measure the angular deviation of collagen fibers as a measure of overall orientation of de novo tissue (60). As illustrated in FIG. 6(A-D), FIG. 7, and FIG. 8, properly oriented sections that are exposing tendon and enthesis longitudinally can be obtained via axial sections. Bone and fibrocartilage markers will be immunostained (type-I coll, type-II coll, COMP, Aggrecan, Osteocalcin) to confirm matrix types.

Timelines and activities are provided in TABLE 2.

TABLE 2
Timelines and activities.

| Activities | Year 1 | | | | Year 2 | | | |
|---|---|---|---|---|---|---|---|---|
| Thread fabrication | X | X | | | | | | |
| Braiding of scaffolds | | X | | | | | | |
| In vitro cadaveric mechanical tests | | | | X | | | | |
| Stage 1 surgery | | | X | X | | | | |
| Muscle stimulation measurements | | | X | X | X | X | X | |
| Stage 2 surgery | | | | | X | X | | |
| Harvest, biomechanics, microCT, | | | | | | X | X | X |
| immuno/histo | | | | | | | | |

### Section 2.2.3: Expected Outcomes, Challenges and Alternative Approaches

The surgical model is close to the previous tendon repair models that we published on with the exception that the trough in the bone is deeper by 4 mm (5, 54). We performed the surgery on one pilot rabbit which affirmed that deeper trough into the trabecular space did not affect the recovery and ambulation of the animal. Therefore, we do not anticipate problems in the surgical model.

We expect functional outcomes of repairs performed by using OsTend scaffolds to exceed those repairs that used monophasic scaffolds. We also expect the biphasic group to demonstrate greater evidence of an enthesis-like regeneration per section 1.2.2.2. Healing biology will involve the presence of a distinct fibrocartilaginous transition at the interface for the TC group as confirmed by immunohistological markers. We also expect the OsTend scaffold group to bear the greater load at smallest footprint among all groups, accordingly, stress bearing capacity of biphasic group will be superior.

If the emerging results from the first set of OsTend scaffold-treated rabbits show low-level of mineralization we may resort to seeding scaffolds with autologous mesenchymal stem cells that are collected at the time of the first surgery to induce chronic defect. We have experience in isolating and proliferating rabbit mesenchymal stem cells and their in vivo delivery with collagen scaffolds (5, 6). We think, though, that there is reasonable likelihood that simpler biomaterial only approach may work given the preliminary data (FIG. 9, FIG. 10 and FIG. 12). In rabbits we operated to date (5, 6) with monophasic ELAC, we have not seen ectopic ossification in the tendon segment. It is possible that, the OsTend scaffold's mineralized osteoconductive segment may result in too exuberant bone formation and expand into tendon domain. Should this be the case in the first few rabbits, we will reduce the amount of mineral deposition at the time of fabrication by subjecting the scaffolds to less cycles of alternating soaking to curb the expansion of mineralization.

### Section 3.0: Data Analysis

In statistical analysis of a single variable, one-way ANOVA will be used (or Kruskal Wallis should Anderson-Darling normality test fails). Post hoc comparisons will be done in the last step using Tukey's post-hoc test (or Mann-Whitney with Bonferroni correction, if non-parametric). Significance will be set at p<0.05 for all studies after adjusting for multiple comparisons. Effect size: For Aim 2 that is testing our overarching hypothesis, the effect size is defined by success benchmarks: biomechanically attainment of material properties that are within 25% those of intact tendon (i.e. three quarters as strong as intact tendon) will define a treatment group that has attained an acceptable level of repair. At this effect size, a sample size of 8 for biomechanical strength, at 15% data scatter will exceed 80% power. The power will be greater than 80% for in vivo joint mechanics measures for which sample size will be N=12.

### LITERATURE CITED

1. Ek, E. T., Neukom, L., Catanzaro, S. & Gerber, C. Reverse total shoulder arthroplasty for massive irreparable rotator cuff tears in patients younger than 65 years old: results after five to fifteen years. Journal of shoulder and elbow surgery/American Shoulder and Elbow Surgeons . . . [et al.] (2013) doi:10.1016/j.jse.2012.11.016.
2. Werner, C. M., Steinmann, P. A., Gilbart, M. & Gerber, C. Treatment of painful pseudoparesis due to irreparable rotator cuff dysfunction with the Delta III reverse-ball-and-socket total shoulder prosthesis. The Journal of bone and joint surgery. American volume 87, 1476-86 (2005).
3. Younesi, M., Islam, A., Kishore, V., Anderson, J. M. & Akkus, O. Tenogenic Induction of Human MSCs by Anisotropically Aligned Collagen Biotextiles. Adv Funct Mater 24, 5762-5770 (2014).
4. Islam, A., Younesi, M., Mbimba, T. & Akkus, O. Collagen Substrate Stiffness Anisotropy Affects Cellular Elongation, Nuclear Shape, and Stem Cell Fate toward Anisotropic Tissue Lineage. Adv Healthc Mater 5, 2237-47 (2016).
5. Learn, G. D. et al. Woven collagen biotextiles enable mechanically functional rotator cuff tendon regeneration during repair of segmental tendon defects in vivo. J Biomed Mater Res B Appl Biomater 107, 1864-1876 (2019).
6. McClellan, P. et al. Mesenchymal Stem Cell Delivery via Topographically Tenoinductive Collagen Biotextile Enhances Regeneration of Segmental Tendon Defects. Am J Sports Med 3635465221097939 (2022) doi:10.1177/03635465221097939.
7. Minns Lowe, C. J., Moser, J. & Barker, K. Living with a symptomatic rotator cuff tear ‘bad days, bad nights’: a qualitative study. BMC Musculoskelet Disord 15, 228 (2014).
8. Rincon-Hurtado, A. M., Rocha-Buelvas, A., Lopez-Cardona, A. & Martinez, J. W. Health-related quality of life of patients with rotator cuff injuries, Cofee Triangle, Colombia, 2013. Rev Bras Ortop 53, 364-372 (2018).
9. S Dewan. Sports Medicine: Technologies and Global Markets: HLC183A|BCC Research. https://www.bccresearch.com/market-research/healthcare/sports-medicine-technologies-markets-report.html.
10. Gialanella, B. & Prometti, P. Effects of corticosteroids injection in rotator cuff tears. Pain Med 12, 1559-65 (2011).
11. Armstrong, A. D., Wiater, J. W. & Athwal, G. S. Rotator Cuff Tears: Surgical Treatment Options. https://orthoinfo.aaos.org/en/treatment/rotator-cuff-tears-surgical-treatment-options/(2017).
12. Gillespie, R. J., Knapik, D. M. & Akkus, O. Biologic and Synthetic Grafts in the Reconstruction of Large to Massive Rotator Cuff Tears. J Am Acad Orthop Surg 24, 823-828 (2016).
13. Metcalfe, A. et al. Subacromial balloon spacer for irreparable rotator cuff tears of the shoulder (START:REACTS): a group-sequential, double-blind, multicentre randomised controlled trial. The Lancet 399, 1954-1963 (2022).
14. Ho, J. Y. & Miller, S. L. Allografts in the treatment of athletic injuries of the shoulder. Sports Med Arthrosc Rev 15, 149-157 (2007).
15. Donaldson, J. et al. Latissimus dorsi tendon transfers for rotator cuff deficiency. Int J Shoulder Surg 5, 95-100 (2011).
16. Hirakawa, Y. et al. The accelerated effect of recombinant human bone morphogenetic protein 2 delivered by beta-tricalcium phosphate on tendon-to-bone repair process in rabbit models. J Shoulder Elbow Surg 27, 894-902 (2018).
17. Ma, C. B. et al. Bone morphogenetic proteins-signaling plays a role in tendon-to-bone healing: a study of rhBMP-2 and noggin. Am J Sports Med 35, 597-604 (2007).
18. Kim, J. G. et al. Enhancement of tendon-bone healing with the use of bone morphogenetic protein-2 inserted into the suture anchor hole in a rabbit patellar tendon model. Cytotherapy 16, 857-67 (2014).
19. Seeherman, H. J. et al. rhBMP-12 accelerates healing of rotator cuff repairs in a sheep model. J Bone Joint Surg Am 90, 2206-19 (2008).
20. Kabuto, Y. et al. Stimulation of Rotator Cuff Repair by Sustained Release of Bone Morphogenetic Protein-7 Using a Gelatin Hydrogel Sheet. Tissue Eng Part A 21, 2025-33 (2015).
21. Hashimoto, Y., Yoshida, G., Toyoda, H. & Takaoka, K. Generation of tendon-to-bone interface ‘enthesis' with use of recombinant BMP-2 in a rabbit model. J Orthop Res 25, 1415-24 (2007).
22. Lee, K. W. et al. Effective healing of chronic rotator cuff injury using recombinant bone morphogenetic protein-2 coated dermal patch in vivo. J Biomed Mater Res B Appl Biomater 105, 1840-1846 (2017).
23. Rickert, M., Jung, M., Adiyaman, M., Richter, W. & Simank, H. G. A growth and differentiation factor-5 (GDF-5)-coated suture stimulates tendon healing in an Achilles tendon model in rats. Growth Factors 19, 115-26 (2001).
24. Murray, D. H. et al. The effect of cartilage-derived morphogenetic protein 2 on initial healing of a rotator cuff defect in a rat model. J Shoulder Elbow Surg 16, 251-4 (2007).
25. Arimura, H. et al. TGF-beta1 Improves Biomechanical Strength by Extracellular Matrix Accumulation Without Increasing the Number of Tenogenic Lineage Cells in a Rat Rotator Cuff Repair Model. Am J Sports Med 45, 2394-2404 (2017).
26. Manning, C. N. et al. Sustained delivery of transforming growth factor beta three enhances tendon-to-bone healing in a rat model. J Orthop Res 29, 1099-105 (2011).
27. Yoon, J. P. et al. Sustained Delivery of Transforming Growth Factor beta1 by Use of Absorbable Alginate Scaffold Enhances Rotator Cuff Healing in a Rabbit Model. Am J Sports Med 46, 1441-1450 (2018).
28. Ide, J. et al. The effect of a local application of fibroblast growth factor-2 on tendon-to-bone remodeling in rats with acute injury and repair of the supraspinatus tendon. J Shoulder Elbow Surg 18, 391-8 (2009).
29. Tokunaga, T. et al. Enhancement of rotator cuff tendon-bone healing with fibroblast growth factor 2 impregnated in gelatin hydrogel sheets in a rabbit model. J Shoulder Elbow Surg 26, 1708-1717 (2017).
30. Tokunaga, T. et al. FGF-2 Stimulates the Growth of Tenogenic Progenitor Cells to Facilitate the Generation of Tenomodulin-Positive Tenocytes in a Rat Rotator Cuff Healing Model. Am J Sports Med 43, 2411-22 (2015).
31. Zhao, S. et al. Biological augmentation of rotator cuff repair using bFGF-loaded electrospun poly(lactide-co-glycolide) fibrous membranes. Int J Nanomedicine 9, 2373-85 (2014).
32. Younesi, M., Islam, A., Kishore, V., Panit, S. & Akkus, O. Fabrication of compositionally and topographically complex robust tissue forms by 3D-electrochemical compaction of collagen. Biofabrication 7, 035001 (2015).
33. Gurkan, U. A., Cheng, X., Kishore, V., Uquillas, J. A. & Akkus, O. Comparison of morphology, orientation, and migration of tendon derived fibroblasts and bone marrow stromal cells on electrochemically aligned collagen constructs. Journal of biomedical materials research. Part A 94, 1070-9 (2010).
34. Kishore, V. et al. In vivo response to electrochemically aligned collagen bioscaffolds. Journal of biomedical materials research. Part B, Applied biomaterials (2011) doi:10.1002/jbm.b.31962.
35. Uquillas, J. A. & Akkus, O. Modeling the electromobility of type-I collagen molecules in the electrochemical fabrication of dense and aligned tissue constructs. Annals of biomedical engineering 40, 1641-53 (2012).
36. Uquillas, J. A., Kishore, V. & Akkus, O. Genipin crosslinking elevates the strength of electrochemically aligned collagen to the level of tendons. J Mech Behav Biomed 15, 176-189 (2012).
37. Kishore, V., Bullock, W., Sun, X., Van Dyke, W. S. & Akkus, O. Tenogenic differentiation of human MSCs induced by the topography of electrochemically aligned collagen threads. Biomaterials 33, 2137-44 (2012).
38. Cheng, X. et al. An electrochemical fabrication process for the assembly of anisotropically oriented collagen bundles. Biomaterials 29, 3278-88 (2008).
39. Brightman, A. O. et al. Time-lapse confocal reflection microscopy of collagen fibrillogenesis and extracellular matrix assembly in vitro. Biopolymers 54, 222-34 (2000).
40. Parry, D. A. The molecular and fibrillar structure of collagen and its relationship to the mechanical properties of connective tissue. Biophys Chem 29, 195-209 (1988).
41. Akkus, O., Kishore, V., Younesi, M. & Islam, A. U.S. patent Ser. No. 10/017,868-Electrochemical processing of materials, methods and production. (2018).
42. Chapin, K. et al. In vivo biocompatibility and time-dependent changes in mechanical properties of woven collagen meshes: A comparison to xenograft and synthetic mid-urethral sling materials. J Biomed Mater Res B Appl Biomater 107, 479-489 (2019).
43. Islam, A., Mbimba, T., Younesi, M. & Akkus, O. Effects of substrate stiffness on the tenoinduction of human mesenchymal stem cells. Acta Biomater 58, 244-253 (2017).
44. Tong, W. Y. et al. Functional replication of the tendon tissue microenvironment by a bioimprinted substrate and the support of tenocytic differentiation of mesenchymal stem cells. Biomaterials 33, 7686-98 (2012).
45. Barber, J. G., Handorf, A. M., Allee, T. J. & Li, W. J. Braided nanofibrous scaffold for tendon and ligament tissue engineering. Tissue Eng Part A 19, 1265-74 (2013).
46. Islam, A. et al. Biomechanical evaluation of a novel suturing scheme for grafting load-bearing collagen scaffolds for rotator cuff repair. Clin Biomech (Bristol, Avon) 30, 669-75 (2015).
47. Tarafder, S. et al. In situ tissue engineering of the tendon-to-bone interface by endogenous stem/progenitor cells. Biofabrication 12, 015008 (2019).
48. Lipner, J. et al. In Vivo Evaluation of Adipose-Derived Stromal Cells Delivered with a Nanofiber Scaffold for Tendon-to-Bone Repair. Tissue Eng Part A 21, 2766-74 (2015).
49. Derwin, K. A., Baker, A. R., Iannotti, J. P. & McCarron, J. A. Preclinical models for translating regenerative medicine therapies for rotator cuff repair. Tissue Eng Part B Rev 16, 21-30.
50. Funakoshi, T. et al. Application of tissue engineering techniques for rotator cuff regeneration using a chitosan-based hyaluronan hybrid fiber scaffold. Am J Sports Med 33, 1193-201 (2005).
51. Yokoya, S., Mochizuki, Y., Nagata, Y., Deie, M. & Ochi, M. Tendon-bone insertion repair and regeneration using polyglycolic acid sheet in the rabbit rotator cuff injury model. Am J Sports Med 36, 1298-309 (2008).
52. Grumet, R. C., Hadley, S., Diltz, M. V., Lee, T. Q. & Gupta, R. Development of a new model for rotator cuff pathology: the rabbit subscapularis muscle. Acta Orthop 80, 97-103 (2009).
53. McClellan, P. E. et al. Volumetric MicroCT Intensity Histograms of Fatty Infiltration Correlate with the Mechanical Strength of Rotator Cuff Repairs: An Ex Vivo Rabbit Model. Clinical Orthopaedics and Related Research® 479, 406-418 (2021).
54. Ina, J et al. Bulk Tendon Regeneration In Electrochemically Aligned Collagen Scaffolds In A Rabbit Rotator Cuff Model. in Orthopaedic Research Society (2021).
55. Gelenitis, K. T., Sanner, B. M., Triolo, R. J. & Tyler, D. J. Selective Nerve Cuff Stimulation Strategies for Prolonging Muscle Output. IEEE Trans Biomed Eng 67, 1397-1408 (2020).
56. Valencia, A. P., Iyer, S. R., Pratt, S. J. P., Gilotra, M. N. & Lovering, R. M. A method to test contractility of the supraspinatus muscle in mouse, rat, and rabbit. J Appl Physiol (1985) 120, 310-317 (2016).
57. Ramasamy, J. G. & Akkus, O. Local variations in the micromechanical properties of mouse femur: the involvement of collagen fiber orientation and mineralization. Journal of biomechanics 40, 910-8 (2007).
58. Loppini, M. et al. Histopathological scores for tissue-engineered, repaired and degenerated tendon: a systematic review of the literature. Curr Stem Cell Res Ther 10, 43-55 (2015).
59. Watkins, J. P., Auer, J. A., Gay, S. & Morgan, S. J. Healing of surgically created defects in the equine superficial digital flexor tendon: collagen-type transformation and tissue morphologic reorganization. Am J Vet Res 46, 2091-2096 (1985).
60. Thomopoulos, S., Marquez, J. P., Weinberger, B., Birman, V. & Genin, G. M. Collagen fiber orientation at the tendon to bone insertion and its influence on stress concentrations. J Biomech 39, 1842-51 (2006).

What is claimed is:

1. A biphasic osteotendinous repair scaffold comprising:
a first phase comprising electrochemically aligned collagen (ELAC) threads and a calcium phosphate mineral and having a major surface; and
a second phase comprising ELAC threads and having a major surface,
wherein:
the ELAC threads of the first phase are braided and crosslinked;
the ELAC threads of the second phase are braided and crosslinked;
the ELAC threads of the first and second phases form first and second interconnected macroporosities throughout the first and second phases, respectively;
the calcium phosphate mineral of the first phase is distributed on the major surface of the first phase and within the interconnected macroporosity of the first phase; and
the second phase is adjacent to the first phase.

2. The scaffold according to claim 1, further wherein:
the ELAC threads of the first phase are twisted to form a yarn;
the ELAC threads of the second phase are twisted to form a yarn;
the yarn of the first phase is braided, thereby forming the ELAC threads of the first phase that are braided; and
the yarn of the second phase is braided, thereby forming the ELAC threads of the second phase that are braided.

3. The scaffold according to claim 2, further wherein:
the yarn of the first phase comprises a 4-ply yarn and a 2-ply yarn, the 4-ply yarn of the first phase is used as triaxial threads within the first phase, and the 2-ply yarn of the first phase is used as oblique threads within the first phase; and
the yarn of the second phase comprises a 4-ply yarn and a 2-ply yarn, the 4-ply yarn of the second phase is used as triaxial threads within the second phase, and the 2-ply yarn of the second phase is used as oblique threads within the second phase.

4. The scaffold according to claim 2, further wherein three or more concentric layers of the yarn of the first phase are braided on top of each other successively, and three or more concentric layers of the yarn of the second phase are braided on top of each other successively.

5. The scaffold according to claim 1, further wherein:
the ELAC threads of the first phase are braided as monofilaments; and
the ELAC threads of the second phase are braided as monofilaments.

6. The scaffold according to claim 1, wherein the scaffold has a load-bearing axis, and a majority of the ELAC threads are positioned along the load-bearing axis.

7. The scaffold according to claim 1, wherein the ELAC threads of the first and second phases are crosslinked with an iridoid crosslinking agent.

8. The scaffold according to claim 7, wherein the iridoid crosslinking agent comprises one or more of genipin, loganin aglycone, oleuropein aglycone, or E-6-O-methoxycinnamoyl scandoside methyl ester aglycone.

9. The scaffold according to claim 7, wherein the iridoid crosslinking agent comprises genipin.

10. The scaffold according to claim 1, wherein the calcium phosphate mineral comprises osteoinductive hydroxyapatite crystals or dicalcium phosphate dihydrate crystals.

11. The scaffold according to claim 1, wherein the second phase is adjacent to the first phase based on being contiguous with the first phase.

12. The scaffold according to claim 1, wherein the second phase is adjacent to the first phase based on being attached directly or indirectly to the first phase.

13. The scaffold according to claim 1, further comprising mesenchymal stem cells seeded on the ELAC threads of the first phase.

14. The scaffold according to claim 13, wherein the mesenchymal stem cells are autologous mesenchymal stem cells.

15. The scaffold according to claim 13, wherein to the extent that human or animal cells other than mesenchymal stem cells are present on the ELAC threads of the first phase, more mesenchymal stem cells are present than the other human or animal cells.
